(19) United States
(12) Patent Application Publication
(10) Pub. No.: US 2006/0035280 A1
(43) Pub. Date: Feb. 16, 2006

(54) Screening methods for inhibitors of protein-protein binding interactions

(76) Inventors: Kuo-Sen Huang, Livingston, NJ (US); Lyubomir Vassilev, North Caldwell, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 11/034,312

(22) Filed: Jan. 12, 2005

Related U.S. Application Data
(60) Provisional application No. 60/536,560, filed on Jan. 15, 2004.

(51) Int. Cl. G01N 33/53

(52) U.S. Cl. 435/7.1

(57) ABSTRACT

Protein-protein interactions represent a large and important group of drug targets involved in the development and progression of human diseases. However, their utilization in drug discovery has been hampered by the low probability of identifying small-molecule inhibitors able to disrupt protein binding with desirable potency and selectivity. Therefore, the capability for rapid screening of large compound libraries has been critical for the exploration of this target class. The present invention relates to a homogeneous time-resolved fluorescence assay for identification of inhibitors of Cks1-Skp2 binding that plays a critical role in the ubiquitin-dependent degradation of p27. The assay was implemented in a 1536-well format using the new Zeiss uHTS robot and achieved a throughput in excess of 100,000 data points per day. A protocol for a fully automated high throughput IC₅₀ determination was developed for hit validation. The basic 1536 well screening platform reported here is simple, robust and cost effective. It is widely applicable to any protein-protein interaction of therapeutic interest.

### PRIORITY TO RELATED APPLICATIONS

This application claims the benefit of Provisional Application Ser. No. 60/536,560, filed Jan. 15, 2004.

### BACKGROUND OF THE INVENTION

The present invention generally relates to methods for screening for compounds that may modulate a protein-protein interaction. In one example, the present invention relates to a high density ultra high throughput screening (uHTS) method for finding inhibitors of the protein-protein binding between p27 ubiquitin ligase subunit Skp2 and Cks1.

The remarkable progress in the understanding of the molecular basis of human disease in the last decade has led to a significant increase in the number of potential therapeutic targets. Among these, protein-protein interactions represent a large and growing class of molecular targets with importance that spans across many therapeutic areas. However, due to the relatively large interacting surfaces involved, these targets have been considered high risk and their exploration is still it its infancy. One way to balance the risk and increase the chance of success in this target class is to design assays that permit rapid screening of large compound libraries.

The natural cyclin-dependent kinase (CDK) inhibitor p27^{Kip1} plays an important role in the regulation of multiple fundamental cellular processes, including proliferation, differentiation, and apoptosis (1, 2). A large number of studies have demonstrated that loss or decreased level of p27 in cancer cells is associated with poor prognosis (3-5) and p27 has been accepted as a an independent prognostic indicator in many major solid malignancies (6). Cellular level of p27 is regulated at the post-translation level by ubiquitin-dependent proteolysis (7). Studies in colorectal, breast, stomach, and lung cancers have shown that the low level of p27 in cancer cells is due to enhanced degradation (6). Therefore, restoration of p27 in cancer cells by inhibiting its ubiquitin-dependent degradation could offer a novel approach to cancer therapy.

p27 is ubiquitinated by a multiprotein complex known as SCF^{skP2} (E3 ubiquitin ligase for p27). The process is initiated with the phosphorylation of p27 on Thr187 by CDK2/cyclin E (8). Phosphorylated p27 is recognized by Skp2, an F-box protein and component of SCF that is specific for p27. Recent studies have demonstrated that the binding of the small nuclear protein Cks1 to Skp2 greatly increases its affinity to p27 and allows for efficient ubiquitination of the protein (9, 10). It has been shown that Cks1 is critical for the ubiquitination of p27 both in vitro and in vivo (10). Based on these studies, Cks1 appears to function as an accessory factor of SCF^{Skp2} that can regulate the ubiquitination of p27. According to this model, the disruption of Cks1-Skp2 protein complex should result in inhibition of p27 degradation.

As can be seen, there exists a need to provide an ultra high throughput screening assay for Cks1-Skp2 inhibitors.

### SUMMARY OF THE INVENTION

According to one embodiment of the present invention, a method for the high-throughput screening of compounds as an inhibitor of a protein-protein binding interaction between p27 ubiquitin ligase subunit Skp2 and Cks1, comprises adding a plurality of test compounds each into separate wells of a well plate; adding a FLAG-Cks1 solution to at least a first number of the wells; adding a GST-Skp2 solution to at least a second number of the wells, wherein the first number includes at least one of the second number; incubating the well plate; adding a Eu-labeled anti-FLAG antibody and an APC-labeled anti-GST antibody to the well plate; incubating the well plate; and reading the well plate to determine whether the compound is an inhibitor of the protein-protein binding interaction between Skp2 and Cks1.

### BRIEF DESCRIPTION OF THE DRAWINGS

FIG. 1 is a diagram of Zeiss uHTS Modules. Each plate lift has 12 stackers and each stacker is capable of holding 24×384-well plates (without lid) or 28×1536-well plates (BD Falcon, Low Base with lid). Cytomat Incubator (Kendro Laboratory Products, CT) is capable of handling 189 plates with temperature (10-50° C.), CO₂ and humidity control. EMBLA washer and Multidrop dispenser were manufactured by Skatron Instruments (Norway) and Labsystems (Finland), respectively. Cybi-well pipettor equipped with Cybi-drop dispenser was made by CyBio (Germany). Multimode Reader with 96 channels is capable of reading absorbance, fluorescence and luminescense (Carl Zeiss, Germany). Microtiter plates can be transported between modules by a conveyer belt and delivered to individual components in the module by turn tables (TT1 or TT2). They can also be disposed by the conveyer belt at both ends (shown as “W”);

FIG. 2 is the principle scheme of the Skp2-Cks1 binding assay. Arrow indicates fluorescence energy transfer between Eu chelate and APC;

FIG. 3 is a diagram showing the performance of the 384 and 1529 assay formats;

FIG. 4 is a flowchart showing the assay process on the Zeisss uHTS system. SP1-SP4 represent 384 well compound plates. AP 1536 represents 1536-well assay plate. Abase is ActivityBase;

FIG. 5 is a flowchart showing data export and analysis according to the present invention, including (A) Data handling process, and (B) Conversion of 1536 matrix files to 384 files for loading to Activity Base;

FIG. 6 is a graph showing the Z' factor and signal to background (S/B) ratios for all the screen plates;

FIG. 7 is a graph showing the Z' factor; and

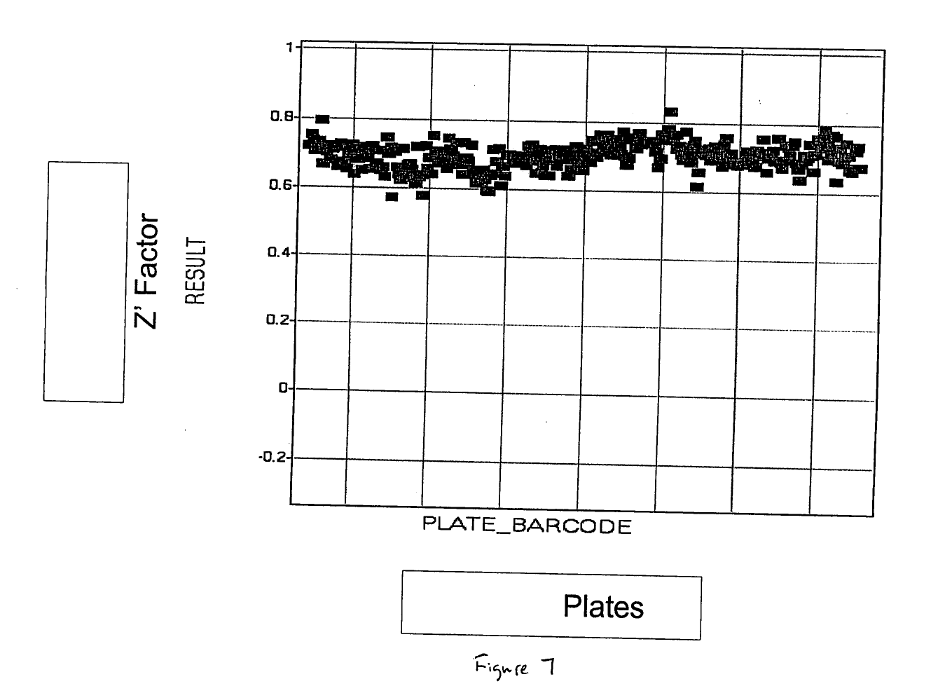

FIG. 8 is an example of an IC50 curve generated by the present invention.

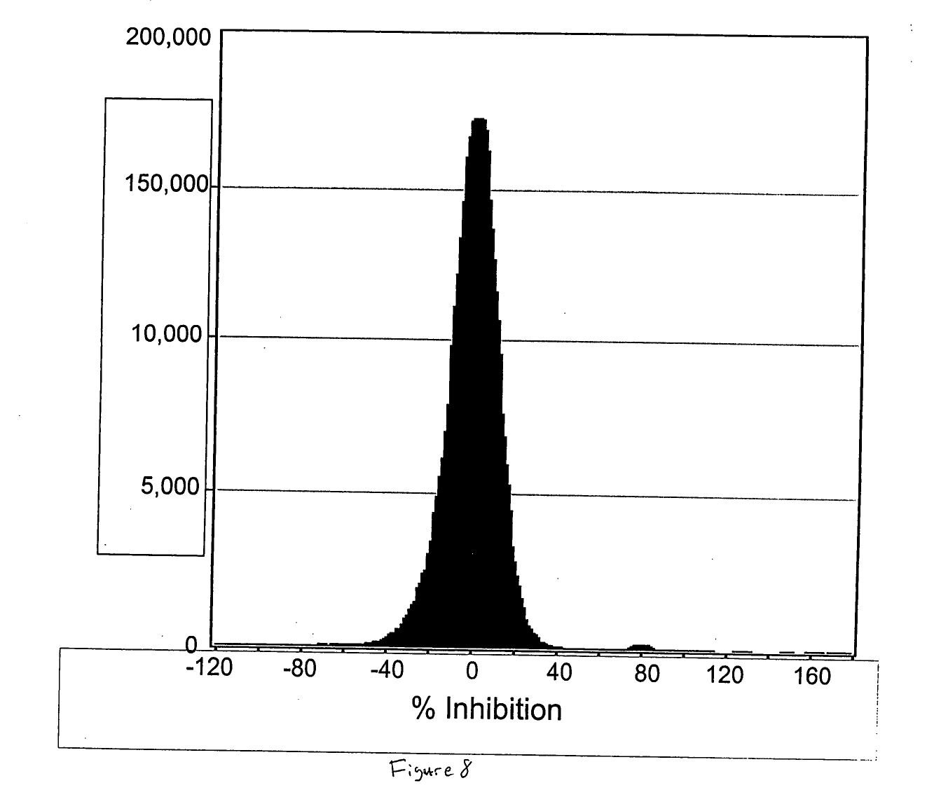

### DETAILED DESCRIPTION OF THE INVENTION

### MATERIALS AND METHODS

Europium-labeled anti-FLAG antibody was purchased from Perkin-Elmer Wallac (Gaithersburg, Md.). Allophycocyanin (APC)-labeled anti-GST antibody was from Prozyme (San Leandro, Calif.). Expression plasmid for GST-Skp2 and Skp1 was constructed in a dicistronic vector (pET/GST-Skp2-Skp1) as described (11,12). Plasmid DNA was transformed into BL21 (DE3) E.coli cells for protein expression and GST-skp2/skp1 complex was purified as described (11). Cks1 was purified as described previously (13).

Skp2-Cks1 Binding Assay in 1536 Well Format

Initially, Skp2-Cks1 HTRF assay was carried out in 1536-well format manually. FLAG-Cks1 (3 μl/well, 3 μg/ml) in Assay Buffer (20 mM Tris-HCl, pH 7.5, 180 mM NaCl, 0.075% BSA and 1 mM DTT) was mixed with 1.5 μl/well of 20 mM Tris-HCl, pH 7.5, 180 mM NaCl and 1 mM DTT followed by 1.5 μl/well of GST-Skp2 (40 μg/ml) in Assay Buffer and incubated at 37° C. for 30 min. Eu-labeled anti-FLAG antibody and APC-anti-GST antibody were premixed in 20 mM Tris-HCl, pH 7.5, 180 mM NaCl and 0.075% BSA to final concentrations of 3 nM and 100 nM, respectively. The antibody solution (2 μl/well) was then added to the assay sample and incubated at room temperature for 30 min and read on Victor 5 (Wallac).

The Zeiss uHTS System

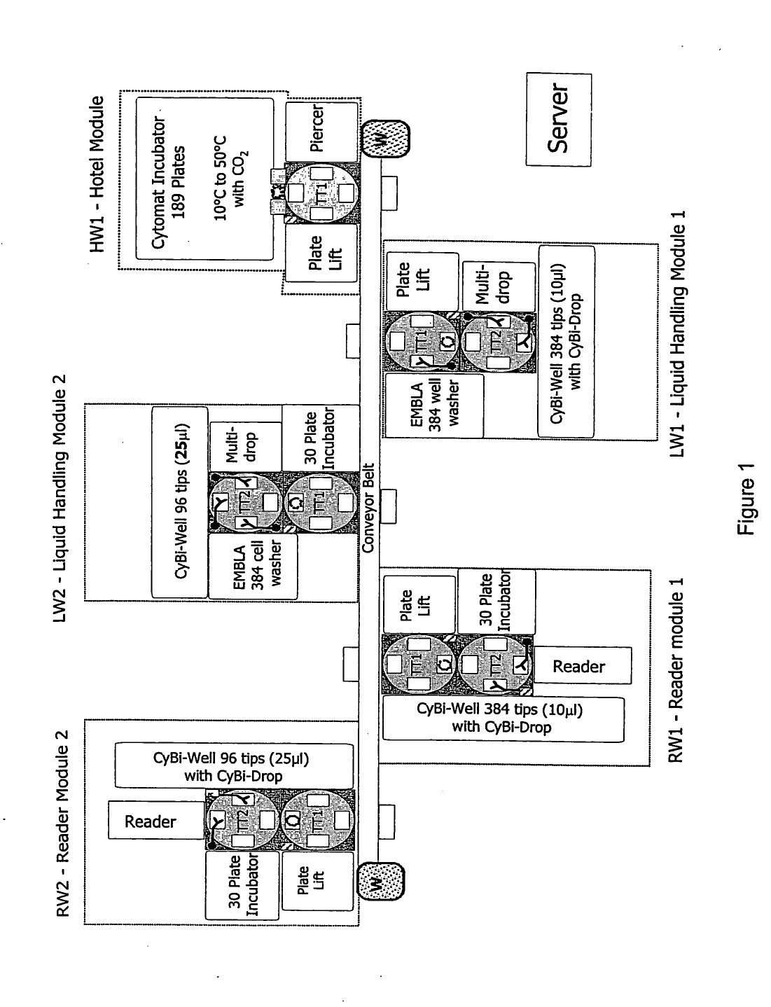

FIG. 1 shows a diagram of a 5-module Zeiss uHTS system. Hotel module (HW1) contains a temperature, CO₂ and humidity controlled incubator capable of holding 189 microtitter plates. In addition, it has a Plate Lift with 12 holders, each capable of holding 24-28 different types of plates. Available Piercer can punch holes through the seals of compound plates, which are usually sealed with aluminum foil to prevent moisture accumulation. Each module contains 1 or 2 turn tables for delivering plates into its components. Liquid Handling Module 1 (LW1) is equipped with a Cybi-Well (384 tips) pipettor with Cybi-Drop for handling small volume (1-10 ul), a Multidrop for dispensing larger volumes (>10 ul), an EMBLA-384 washer and a Plate Lift. Liquid Handling module 2 (LW2) is equipped with a CyBi-Well (96 tips) pipettor, multidrop, EMBLA 384 cell washer and a 30-plate incubator (25-50° C.). Reader Module 1 (RW1) contains a Cybi-Well (384 tips), a Plate Lift, a 30-plate incubator and a Zeiss Multimode Reader capable of reading absorbance, fluorescence, luminescence, and fluorescence polarization, but not time-resolved fluorescence^{a}. Reader Module 2 (RW2) contains the same components as RW1 except the Cybi-Well (96 tips) pipettor.

Compound plates or assay plates are placed in the Plate Lift. They are picked up by a robotic arm and placed on a turn table. They can be delivered to individual components by turn tables that have pusher device to push plates back and forth between turn tables and individual components. Turn tables have a flipper device to turn plates around for correct orientation. In addition, turn tables have a vacuum-driven device for removing and replacing lids. Plates are transported between modules by a conveyor belt. They can be wasted at either end by the conveyor belt. Each module has its own computer and can be operated as an independent workstation. There is a server computer that controls multi-module operations. The software program (“PlateWork”) has features for maximum throughput operation and equal time treatment for individual plates.

Implementation of Skp2-Cks1 Assay

Polypropylene 384-well plates containing testing compounds from columns 3 to 24 (2 μl per well, 1 mM in DMSO) were placed in the HW1 plate-lift and polystyrene 1536-well assay plates were placed in LW1 plate-lift. An assay plate was lifted by a robotic arm, placed in a turn-table, and moved to the Cybi-well 384 pipettor in LW1 FLAG-Cks1 (3 μl/well, 3.0 μg/ml) in 20 mM Tris-HCl, pH 7.5, 1.80 mM NaCl, 1 mM DTT and 0.075% BSA was added by the Cybi-well 384 pipettor. Compound plates were then transported to the Cybi-drop in LW1 and 20 μl/well of 20 mM Tris-HCl, pH 7.5, 180 mM NaCl, 1 mM DTT was added (columns 1-2 contain assay buffer only and columns 3-24 contain compounds) through the Cybi-drop. After mixing three times with the 384 pipettor, 1.5 μl/well was transferred to the assay plate. Samples from four 384-well compound plates were added to one 1536-well plate. The assay plate was then moved to the Cybi-well 384 pipettor in RW1. GST-Skp2 (1.5 μl/well, 40 μg/ml) in 20 mM Tris-HCl, pH 7.5, 180 mM NaCl, 1 mM DTT and 0.075% BSA was stored in “Smart Trough” (a divided reagent reservoir) and added to columns 5-48. Assay Buffer was added to the top half of wells in column 1-4 as a background. A premixed GST-Skp2 (40 μg/ml) and Cks1 (4.0 μg/ml) solution was added to the bottom half of wells in columns 1-4 as an inhibitor control. The sample was then mixed 3 times using the 384 pipettor and the plate was incubated at 37° C. for 30 min (I30 incubator in RW1).

For FLAG-Cks1/GST-Skp2 complex detection, a premixed Eu-labeled anti-FLAG antibody (3 nM) and APC-anti-GST antibody (100 nM) solution in 20 mM Tris-HCl, pH 7.5, 180 mM NaCl and 0.075% BSA was added to the assay plate (2 μl/well) through the Cybi-well pipettor in RW1 and the plate was incubated at 190° C. in a humidified incubator (I189 incubator) in HW1 for at least 1 hr. The assay plates were centrifuged briefly to remove air bubbles before reading.

HTRF Plate Reader and Data Handling

Assay plates were read on ViewLux reader (PerkinElmer Life Sciences) using excitation at 340 nm and emission at 665 nm (50 μs delay and 354 μs acquisition window) and 615 nm (50 μs delay and 354 μs acquisition window). The instrument was calibrated for the plate configuration using a blank 1536-well microtiter plate (BD Falcon, Cat # 353249) and for the donor spectrum cross-talk at 665 nm using 1 nM of Eu-labeled anti-FLAG antibody in the assay buffer. The output file contains a blank-corrected normalized ratio (Rn) calculated by the following formula:
Rn=[(A−Bₐ−C×D)/(D−B_{d})]×(D_{c}−B_{d})
where A is the fluorescence intensity of the sample at 665 nm
D is the fluorescence intensity of the sample at 615 nm
Bₐ and B_{d} are plate backgrounds at 665 nm and 615 nm, respectively
D_{c} is the fluorescence intensity of 1 nM Eu-labeled anti-FLAG antibody in the assay buffer at 615 nm

The cross talk factor (C) is determined by the following formula:
C=(A_{c}−Bₐ)/(D_{c}−B_{d})
where A_{c} is the fluorescence intensity of 1 nM Eu-labeled anti-FLAG antibody in the assay buffer at 665 nm

The assay data was in 1536-well matrix format and converted to 384-well format by a splitter macro program and entered Activity Base for analysis.

IC₅₀ Determination Protocol

Primary hits (1 mM in DMSO) were added to columns 5 and 15 (16 compounds per column or 32 compounds per plate, 40 μl/well) in a 384-well polypropylene plate and diluted 3 fold in series in DMSO (10 concentrations per compound, columns 5-14 and 15-24) with a Tecan Genesis (8 channel tips). DMSO (20 μl/well) was added to wells in columns 1-4 as controls. Ten plates containing 312 hits were then moved to the Zeiss uHTS system for transferring 2.8 μl/well of compound solution from each plate to another polypropylene plate in 20 mM Tris-HCl, pH 7.5, 180 mM NaCl and 1 mM DTT (30 μl/well) using the Cybi-Well 384 pipettor in LW1. The compound plates were then tested for inhibition of Cks1-Skp2 binding on the Zeiss uHTS system using the same assay protocol as described above except that the initial compound dilution step was omitted. In addition, compounds were testing in quadruplicates by adding aliquots of compound solutions into 4 quadras of the 1536-well of assay plates. Data were entered Activity Base and IC50s were determined by the 4-parameter fit equation. The IC50s of compounds were calculated by the averages of IC50s in quadruplicates.

### RESULTS AND DISCUSSION

Development of a 1536 Well Skp2-Cks1 Binding Assay

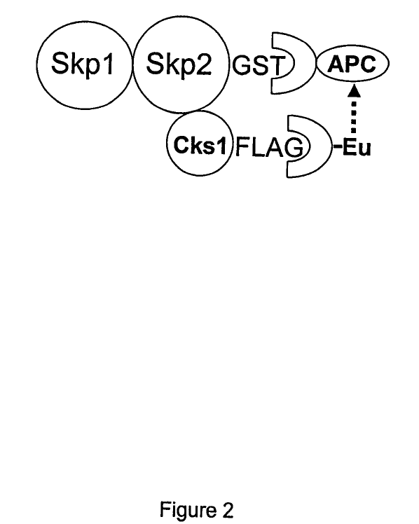

The HTRF binding assay to study the interactions between the F-box protein and component of p27 ubiquitin ligase Skp2 and Cks1 used human recombinant GST-Skp2 and FLAG-Cks1 (11). We have shown that the tags did not interfere with the binding ability of the proteins (11). These generic epitope tags not only helped in the purification of the protein but allowed for designing an assay using commercially available reagent: APC-conjugated anti-GST antibody and Eu-labeled anti-FLAG antibody from Wallac. To increase the yield of recombinant Skp2 the protein was co-expressed and co-purified as an equimolar complex with its natural interacting partner Skp1, a basic component of SCF^{Skp2}. All proteins used in the assay were greater than 90% pure. The amount of bound Skp2 and Cks1 was measured by fluorescence energy transfer (FRET) between Eu and APC as schematically illustrated in FIG. 2.

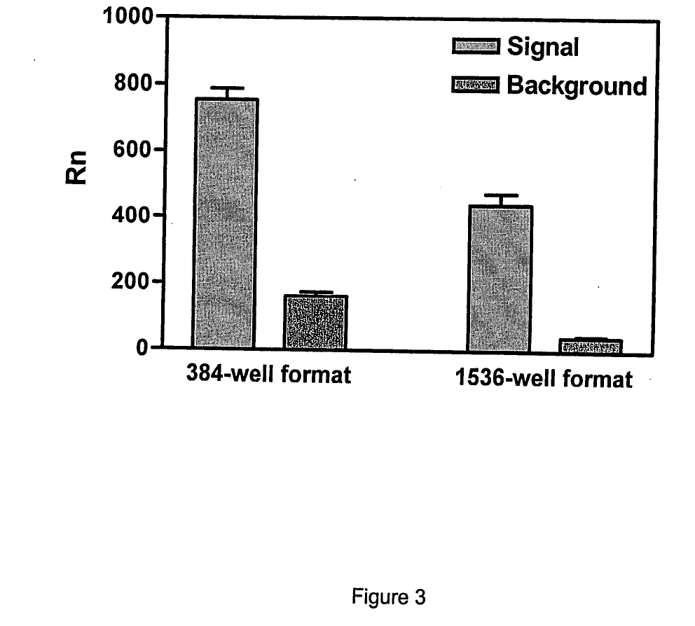

Using this assay in a 384 well format, we investigated the binding affinity between Skp2 and Cks1 and determined a Km of 140 nM. This affinity is in the range that is considered acceptable for development of small-molecule protein-protein binding inhibitors. In addition, the crystal structure of Cks1 suggested that the binding surface contributed by the Cks1 molecule may be limited to a short alpha helix (10,14) which increases the likelihood of identifying small-molecule compounds that can interfere effectively with the interaction. To explore this possibility, we developed a 1536 well version of the assay that can permit ultra high throughput screening for identification of Skp2-Cks1 binding inhibitors. The performance of the 384 and 1529 assay formats is shown in FIG. 3. While the higher density format did not change significantly the Z' factor of the assay (0.77 to 0.72) it increased dramatically its potential throughput and decreased reagent cost per assay approximately 50%.

Implementation and Performance of the Assay on the Zeiss uHTS System

To take full advantage of the potential throughput of the screening assay, it was implemented on the Zeiss uHTS robot. This is a new generation fully automated screening system developed recently by Carl Zeiss (Jena, Germany).

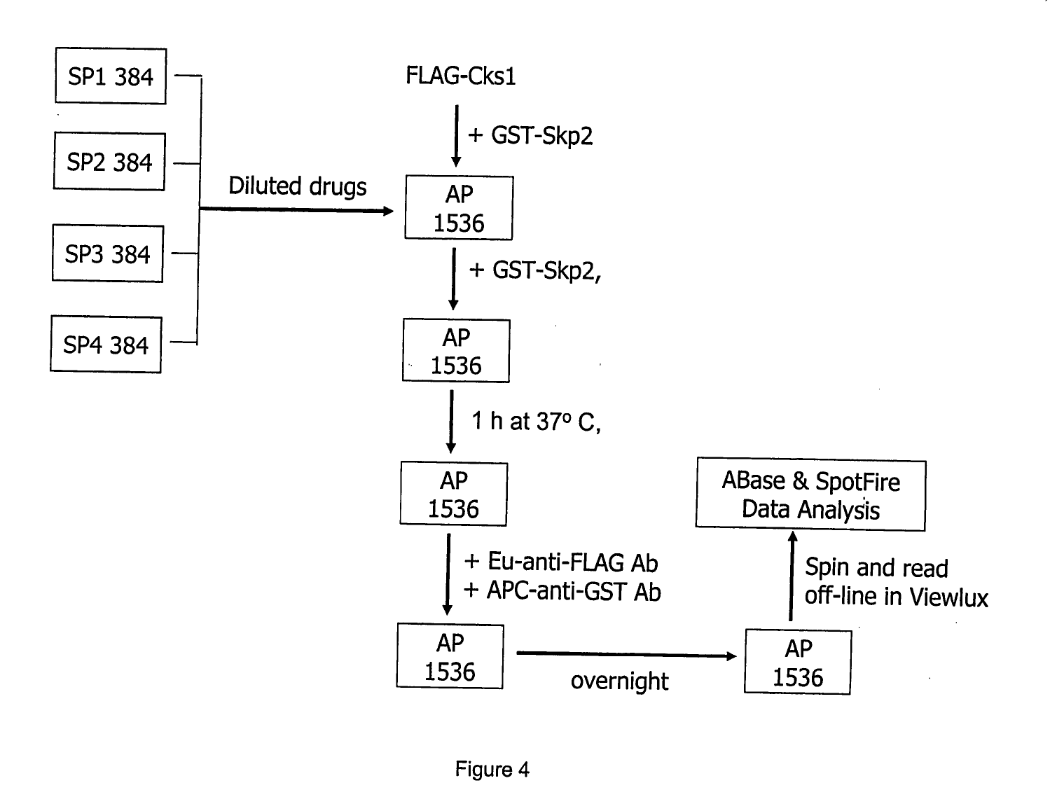

A schematic flow-chart of the assay protocol is shown in FIG. 4. The whole process utilized 3 modules (HW1, LW1 and RW1), and involved 4 pipetting steps, 2 incubation steps, 1 centrifugation step and an off-line read. In the first step, GST-Skp2 solution (ul/well) was dispensed into 1536-well plates by a Cybi-well 384 pipettor (LW1). In order to achieve accurate pipetting of small volumes, the tips needed to be centered in the plate wells at the lowest possible tip-to-bottom distances. This ensured precise delivery of the liquid aided by the surface tension contact with the bottom of the wells. To minimize the formation of air bubbles, reagents and buffers were routinely de-gassed. In addition, we found that it is important to optimize pipetting conditions (e.g. aspirating at medium speed and dispensing at low speed). Due to the very small surface area of each well, blowout of reagents from tips tent to generate air bubbles. We routinely left 0.5 μl liquid in the tips and dispensed back to reagent reservoir or to waste to get accurate mall volume pipetting.

In the second step, compound solutions were added into 1536 well plates. Four compound plates (384-well format) were transferred to one 1536-well assay plate. The assay plate was then transported to RW1 module and FLAG-Cks1 solution as well as controls (background, 100% signal and inhibitor) was added through the Cybi-well 384 dispenser. The plates were incubated at 37° C. for 30 min that allowed Skp2-Cks1 complex formation to reached equilibrium. A premixed solution containing Eu-labeled anti-FLAG antibody and APC-labeled anti-GST antibody was then added and the plates were incubated in a humidified incubator at 19° C. for at least 1 hour or overnight. Although air bubbles were rarely seen at this stage, we routinely centrifuged plates briefly before reading offline on the ViewLux.

In order to minimize the evaporation of the small assay volume (8 ul/well), the assay was carried out using plates with lids. Typically, less than 5% evaporation was observed during the entire assay duration. When the plates were sat for more than 12 hours on overnight runs, we stored them in a humidified incubator at 19° C. However, despite these measures some additional evaporation (approximately 15%) was observed. Nonetheless, the Z' factor was not significantly affected. The Zeiss uHTS system has unique features in handling plates in a parallel process. This significantly increases the throughput. By using the above protocol, we routinely achieved a throughput of 70 assay plates in a period of 18 hours, corresponding to over 107,000 data points.

Data Analysis

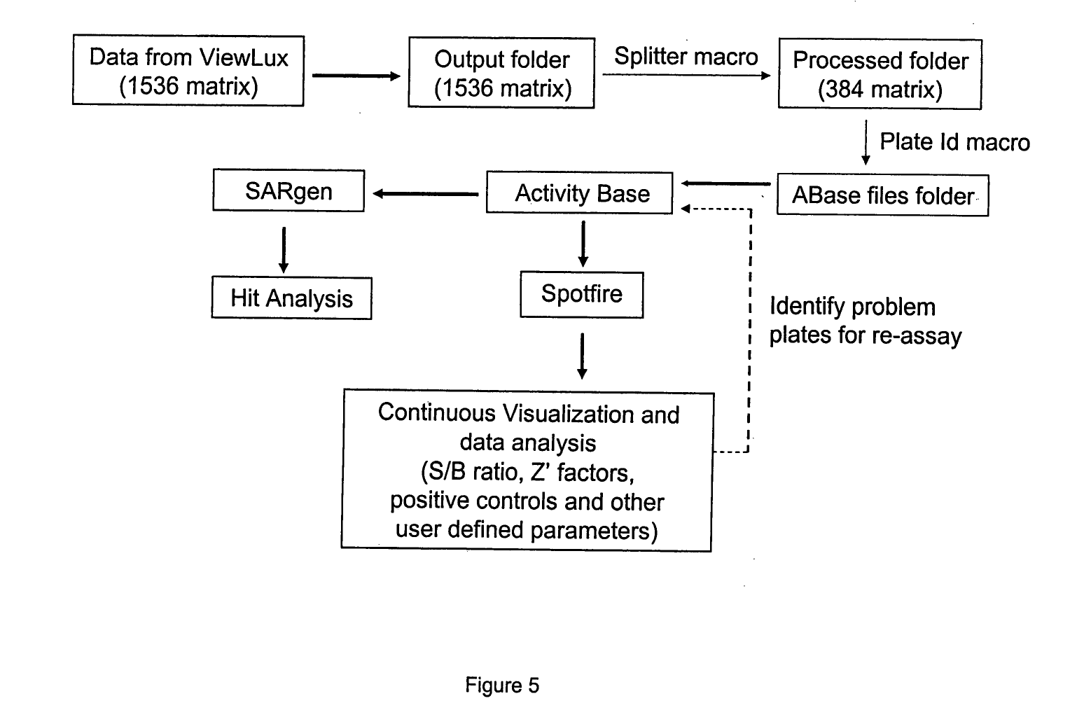

Data handling and analysis is illustrated by a flowchart in FIG. 5. Since the output files from ViewLux are in 1536 well matrix format and compound plates are in 384-well format, we first used a splitter macro to convert a 1536-well file to four 384-well format. Because there is no barcode reader on ViewLux, we used a second macro to align plate IDs with the converted files for loading into data analysis software (Activity Base).

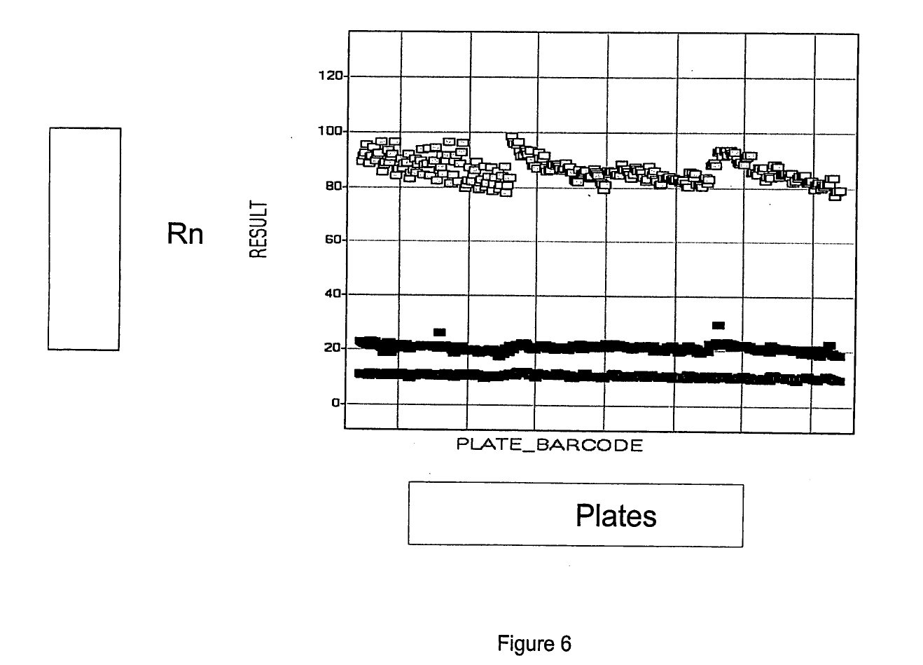

To evaluate the quality of the screen we used the Spotfire software. FIG. 6 shows the Z' factor and signal to background (S/B) ratios for all the screening plates. The Z' factor ranged from 0.5 to 0.7. The screening data was analyzed within 18 hours of testing and this allowed to use the same diluted compound plates to retest plates with Z' factor below 0.5. This quality control step permitted to identify potential problems and minimized the wasting of compound plates.

We developed a 1536 well protocol for IC₅₀ determination of primary hits using the automation capabilities of the Zeiss uHTS system. Primary hits were dispensed into 384 well polypropylene plates in quadruplicates and diluted in series with DMSO using the Tecan Genesis station. Plates were then moved to the Zeiss uHTS robot for testing. Using the above protocol, we routinely generated over 500 IC₅₀ curves per day.

### CONCLUSION

During the past several years, the size of compound libraries has increased dramatically due to the rapid progress in high throughput chemical synthesis. On the other hand, the number of screening targets has been steadily increasing due to advances in the understanding of the molecular basis of diseases. Protein-protein interactions represent one of the major classes of potential drug targets but their exploration has been limited. Assays that permit ultra high throughput screening of large compound libraries at reasonable cost can balance the high risk associated with this target class.

We have developed a generic HTRF-based assay platform for protein-protein interaction that allows ultra high throughput screening in high density formats. It is applicable to most protein-protein interaction that can tolerate epitope tags. The assay platform was tested by screening for inhibitors of the interaction between p27 ubiquitin ligase component Skp2 and Cks1 using the Zeiss uHTS system. The Zeiss robot is one of the newest additions to the arsenal of screening tools which offers full automation of the screening process and the capability to handle high density plate formats. We have developed a 1536 well assay protocol and achieved daily throughput in excess of 100,000 data points. This protocol is robust and reduces screening time and cost by approximately 50% over 384 well format.

### REFERENCES

The following references have been described by number in the above specification. Each of the references discussed above and cited below are hereby incorporated in their entirety by reference for the subject matter which they contain.
1. Reed, S. I., Baillly, E., Dulic, V., Hengst, L., Resnitzky, D. and Slingerland, J. (1994) G1 control in mammalian cells. J. Cell Sci. 18, 69-73.
2. Sherr, C. J. and Roberts, J. M. (1999) CDK inhibitors: positive and negative regulators of G1-phase progression. Genes Dev. 13, 1501-1512.
3. Loda M., Cukor, B., Tam, S. W., Lavin, P., Fiorentino, M., Draetta, G. F., Jessup, J. M. and Pagano, M. (1997) Increased proteasome-dependent degradation of the cyclin-dependent kinase inhibitor p27 in aggressive colorectal carcinomas. Nat. Med. 3, 152-154.
4. Esposito, V., Baldi, A., De Luca, A., Groger, A. M., Loda, M, Giordano, G. G, Caputi, M., Baldi, F., Pagano, M. and Giordano, A. (1997) Prognostic role of the cyclin-dependent kinase inhibitor p27 in non-small cell lung cancer. Cancer Res. 57, 3381-3385.
5. Slingerland, J. and Pagano, M. (2000) Regulation of the cdk inhibitor p27 and its deregulation in cancer. J. Cell Physiol. 183, 10-17.
6. Tsihlias, J., Kapusta, L. and Slingerland, J. (1999) The prognostic significance of altered cyclin-dependent kinase inhibitor in human cancer. Ann. Rev. Med. 50, 401-423.
7. Vlach, J., Hennecke, S. and Amati, B. (1997) Phosphorylation-dependent degradation of the cyclin-dependent kinase inhibitor p27. EMBO J. 16, 5334-5344.
8. Tsvetkov, L. M., Yeh, K. H., Lee, S. J., Sun, H. and Zhang, H. (1999) p27^{Kip1} ubiquitination and degradation is regulated by the SCF^{Skp2} complex through phosphorylated Thr187 in p27. Curr. Biol. 9, 661-664.
9. Ganoth, D., Bornstein, G., Ko, T. K., Larsen, B., Tyers, M., Pagano, M. and Hershko, A. (2001) The cell-cycle regulatory protein Cksl is required for SCF^{Skp2}-mediated ubiquitinylation of p27. Nat. Cell Biol. 3, 321-324.
10. Spruck, C., Strohmaier, H., Watson, M., Smith, A. P., Ryan, A., Krek, T. W. and Reed, S. I. (2001) A CDK-independent function of mammalian Cks1: targeting of SCF^{Skp2} to the CDK inhibitor p27Kip1. Mol. Cell 7,639-650.
11. Xu K., Belunis C, Chu W, Weber D, Podlaski F, Huang K S, Reed S I, Vassilev L T. Protein-protein interactions involved in the recognition of p27 by E3 ubiquitin ligase. Biochem J 2003; 371:957-964.
12. Schulman, B. A., Carrano, A. C., Jeffrey, P. D., Bowen, Z., Kinnucan, E. R., Finnin, M.

S., Elledge, S. J., Harper, J. W., Pagano, M. and Pavletich, N. P. (2000) Insights into SCF ubiquitin ligases from the structure of the Skp1-Skp2 complex. Nature 408, 381-386
13. Arvai, A. S., Bourne, Y., Williams, D., Reed, S. I. and Tainer, J. A. (1995) Crystallization and preliminary crystallographic study of human CksHs1: a cell cycle regulatory protein. Proteins 21, 70-73.
14. Bourne, Y., Watson, M. H., Hickey, M. J., Holmes, W., Rocque, W., Reed, S. I. and Tainer, J. A. (1996) Crystal structure and mutational analysis of the human CDK2 kinase complex with cell cycle-regulatory protein CksHs1. Cell 84, 863-874.

What is claimed is:

1. A method for the high-throughput screening of compounds as an inhibitor of a protein-protein binding interaction between p27 ubiquitin ligase subunit Skp2 and Cks1, comprising:
adding a plurality of test compounds each into separate wells of a well plate;
adding a FLAG-Cks1 solution to at least a first number of said wells;
adding a GST-Skp2 solution to at least a second number of said wells, wherein said first number includes at least one of said second number;
incubating said well plate;
adding a Eu-labeled anti-FLAG antibody and an APC-labeled anti-GST antibody to said well plate;
incubating said well plate; and
reading said well plate to determine whether said compound is an inhibitor of said protein-protein binding interaction between Skp2 and Cks1.
